(19) United States
(12) Patent Application Publication
(10) Pub. No.: US 2012/0078110 A1
(43) Pub. Date: Mar. 29, 2012

(54) ULTRASOUND PROBE

(75) Inventor: Katsuya YAMAMOTO, Ashigara-kami-gun (JP)

(73) Assignee: FUJIFILM Corporation, Tokyo (JP)

(21) Appl. No.: 13/217,655

(22) Filed: Aug. 25, 2011

(30) Foreign Application Priority Data
Sep. 24, 2010 (JP) 2010-213757

(51) Int. Cl. A61B 8/00

(52) U.S. Cl. 600/459

(57) ABSTRACT

An ultrasound probe comprises: a transducer array having an ultrasound transmission/reception surface; a processing circuit mounted on a signal processing board for processing an ultrasonic echo from a subject received with the transducer array to produce a reception signal; a housing including a transducer array compartment for housing the transducer array at one end and the signal processing board near the transducer array, the housing including a projection at another end projecting in a given direction perpendicular to a center line of an arrangement of the transducer array and a grip between the projection and the transducer array compartment for holding from the given direction, a center of gravity lying in a position away from the center line of the arrangement of the transducer array in the given direction when the ultrasound transmission/reception surface of the transducer array is in a horizontal posture.

### BACKGROUND OF THE INVENTION

The present invention relates to an ultrasound probe and particularly to improvement in stability of probe operation.

Conventionally, ultrasound diagnostic apparatus using an ultrasound images have been put to use in the medical field. In general, this type of ultrasound diagnostic apparatus comprises an ultrasound probe having a built-in transducer array and an apparatus body connected to the ultrasound probe. The ultrasound probe transmits ultrasonic waves toward a subject, receives ultrasonic echoes from the subject, and the apparatus body electrically processes the reception signals to generate an ultrasound image.

With such ultrasound diagnostic apparatus, diagnosis is given with the ultrasound probe held in one hand, with the ultrasound transmission/reception surface of the transducer array placed in contact with the surface of a subject. To obtain a high-accuracy ultrasound image, the ultrasound probe needs to be held in a stable posture.

However, since the ultrasound probe has the transducer array disposed at a lowermost position so that the ultrasound transmission/reception surface is exposed. Therefore, when a circuit board for signal processing, a battery for power supply, and the like are provided inside the probe, these built-in components need to be disposed above the transducer array. Then, the center of gravity of the probe is necessarily placed at a higher position, making it difficult to hold the ultrasonic probe in a stable posture.

JP 04-30835 A describes an ultrasound probe using as a filler material provided in the housing of the ultrasound probe a plurality of kinds of filler materials different in density depending on their locations in the probe to place the center of gravity of the probe at a lower position.

JP 2002-65666 A describes an ultrasound probe of which the grip of the housing has a recess to make the grip easy for the operator to hold and thus increase ease of operation.

However, although the center of gravity may be located at a lower position with the ultrasound probe described in JP 04-30835 A, a significant improvement in handling cannot be expected from a mere configuration having the center of gravity at a lower position, because normally this kind of ultrasonic probe is often held from a lateral direction with the ultrasound transmission/reception surface directed downward in operation, and, therefore, holding the ultrasonic probe in a stable posture for a long time period is difficult.

Further, forming a recess in the grip of the housing as in the ultrasound probe described in JP 2002-65666 A does not change the weight bearing on the operator's hand, failing to lighten the burden on the operator.

### SUMMARY OF THE INVENTION

An object of the present invention is to provide an ultrasound probe that resolves such problems of the past and enables ultrasound diagnosis to be given with the probe held in a stable posture.

An ultrasound probe according to the present invention comprises:

a transducer array having an ultrasound transmission/reception surface;

a processing circuit mounted on a signal processing board for processing an ultrasonic echo from a subject received with the transducer array to produce a reception signal;

a housing including a transducer array compartment for housing the transducer array at one end and the signal processing board near the transducer array,

the housing including a projection at another end projecting in a given direction perpendicular to a center line of an arrangement of the transducer array and a grip between the projection and the transducer array compartment for holding from the given direction,

a center of gravity lying in a position away from the center line of the arrangement of the transducer array in the given direction when the ultrasound transmission/reception surface of the transducer array is in a horizontal posture.

### BRIEF DESCRIPTION OF DRAWINGS

FIG. 1 is a block diagram illustrating a configuration of an ultrasound probe according to Embodiment 1 of the invention.

FIG. 2 is a side view illustrating the ultrasound probe according to Embodiment 1 in operation.

FIG. 3 is a top plan view illustrating the ultrasound probe according to Embodiment 1 in operation.

FIG. 4 is a block diagram illustrating an internal configuration of an ultrasound diagnostic apparatus comprising the ultrasound probe according to Embodiment 1.

FIG. 5 is a side view illustrating the ultrasound probe according to Embodiment 2 in operation.

FIG. 6 is a side view illustrating the ultrasound probe according to Embodiment 3 in operation.

FIG. 7 is a top plan view illustrating the ultrasound probe according to Embodiment 3 in operation.

### DETAILED DESCRIPTION OF THE INVENTION

Embodiments of the present invention will be described below based on the appended drawings.

### Embodiment 1

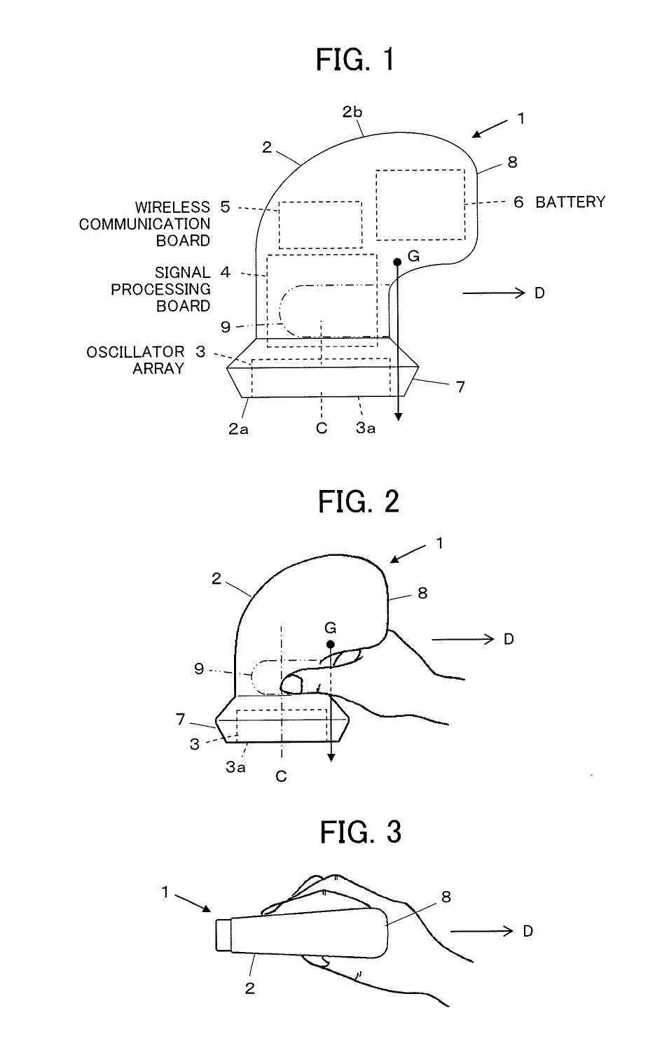

FIG. 1 illustrates an ultrasound probe 1 according to Embodiment 1 of the invention. The ultrasound probe 1 comprises a housing 2 containing a transducer array 3, a signal processing board 4, a wireless communication board 5, and a battery 6.

The housing 2 has at one end portion 2a thereof a transducer array compartment 7 for housing the transducer array 3. The transducer array 3 comprises an ultrasound transmission/reception surface 3a facing outward from the one end portion 2a of the housing 2. The other end portion 2b of the housing 2 has a projection 8 projecting in a given direction D perpendicular to a center line C of the arrangement of the transducer array 3. The projection 8 houses the battery 6 therein.

Further the housing 2 comprises a grip 9 between the transducer array compartment 7 and the projection 8. The grip 9 is provided for the operator to hold the housing 2 from the given direction D in which the projection 8 projects as illustrated in FIGS. 2 and 3.

In the housing 2, the signal processing board 4 is disposed immediately above and close to the transducer array 3 and housed for the most part in the grip 9. The wireless communication board 5 is disposed above the signal processing board 4; the signal processing board 4 and the wireless communication board 5 are positioned generally on the center line C of the arrangement of the transducer array 3.

The battery 6 housed in the projection 8 is disposed in a position significantly away from the center line C of the arrangement of the transducer array 3 in the given direction D. The battery 6 has a significant weight among the components of the ultrasound probe 1 such that the center of gravity G of the whole ultrasound probe 1 lies in a position away from the center line C of the arrangement of the transducer array 3 in the given direction D when the ultrasound transmission/reception surface 3a of the transducer array 3 is placed in a horizontal posture. More specifically, the position of the center of gravity G lies on the outside of the periphery of the grip 9 in the given direction D as illustrated in FIG. 1.

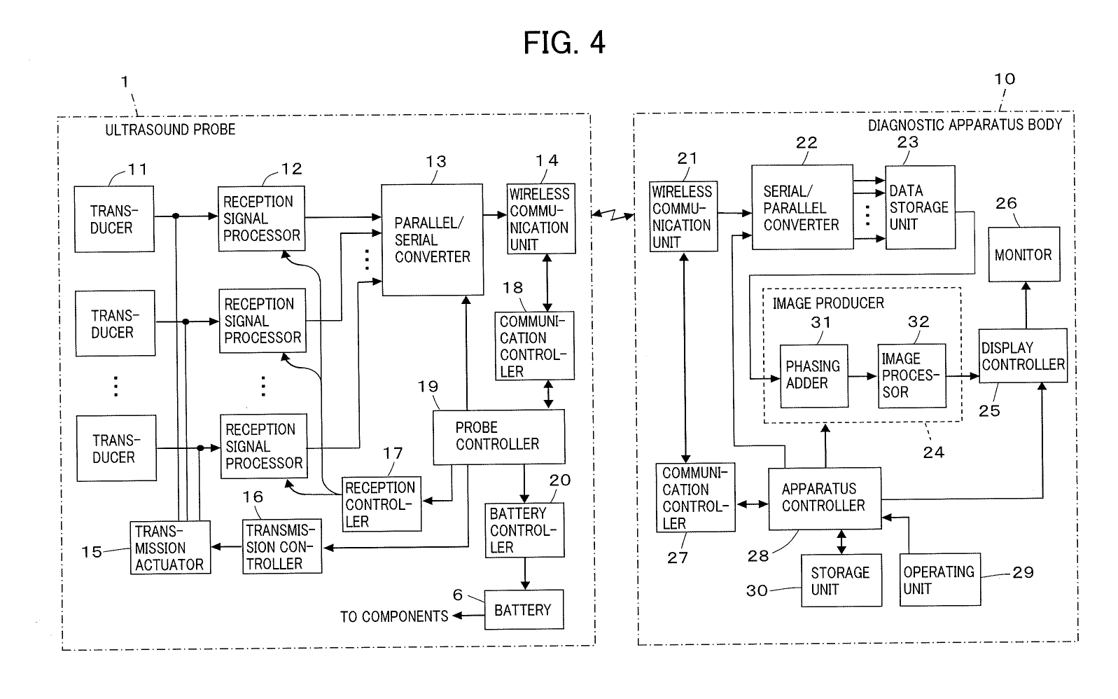

FIG. 4 illustrates an internal configuration of the ultrasound probe 1 according to Embodiment 1. The ultrasound probe 1 is connected to a diagnostic apparatus body 10 by wireless communication.

The ultrasound probe 1 comprises a plurality of ultrasound transducers 11 constituting a unidimensional or two-dimensional transducer array 3, and the transducers 11 are respectively connected to reception signal processors 12, which in turn are connected to a wireless communication unit 14 via a parallel/serial converter 13. The transducers 11 are connected to a transmission controller 16 via a transmission actuator 15, and the reception signal processors 12 are connected to a reception controller 17, while the wireless communication unit 14 is connected to a communication controller 18. The parallel/serial converter 13, the transmission controller 16, the reception controller 17, and the communication controller 18 are connected to a probe controller 19.

The probe controller 19 is connected to a battery 6 via a battery controller 20.

The signal processing board 4 of the ultrasound probe 1 illustrated in FIG. 1 has mounted thereon the reception signal processors 12, the parallel/serial converter 13, the transmission actuator 15, the transmission controller 16, the reception controller 17, the probe controller 19, and the battery controller 20. The wireless communication board 5 has mounted thereon the wireless communication unit 14 and the communication controller 18.

The transducers 11 each transmit ultrasonic waves according to actuation signals supplied from the transmission actuator 15 and receive ultrasonic echoes from the subject to output reception signals. Each of the transducers 11 is composed of a transducer comprising, for example, a piezoelectric body such as a piezoelectric ceramic represented by a PZT (lead zirconate titanate), a polymeric piezoelectric device represented by a monocrystal and a PVDF (polyvinylidene flouride), and the like and electrodes each provided on both ends of the piezoelectric body.

When the electrodes of each of such transducers are supplied with a voltage, which may be in the form of pulse or continuous waves, the piezoelectric body expands and contracts and the transducer generates ultrasonic waves in the form of pulse or continuous waves. These ultrasonic waves are synthesized to form an ultrasonic beam. As each transducer receives propagating ultrasonic waves, it expands and contracts to generate an electric signal and outputs the electric signal as reception signal of the ultrasonic waves.

The transmission actuator 15 comprises, for example, a plurality of pulsers and adjusts the delay amounts of actuation signals for the respective transducers 11 based on a transmission delay pattern selected by the transmission controller 16 so that the ultrasonic waves transmitted from the transducers 11 form a broad ultrasonic beam to cover an area of a tissue of the subject and supplies the transducers 11 with the adjusted actuation signals.

Under the control of the reception controller 17, the reception signal processor 12 on each channel subjects the reception signal outputted from the corresponding transducer 11 to quadrature detection or quadrature sampling process to produce a complex base band signal and samples the complex base band signal to generate sample data containing information on the area of the tissue. The reception signal processors 12 may generate sample data by performing data compression for high-efficiency coding on the data obtained by sampling the complex base band signals.

The parallel/serial converter 13 converts parallel sample data generated by reception signal processors 12 on the plurality of channels into serial sample data.

The wireless communication unit 14 performs carrier modulation according to the serial sample data to generate a transmission signal and supplies an antenna with the transmission signal so that the antenna transmits radio waves to achieve transmission of the sample data. The modulation methods that may be employed herein include ASK (Amplitude Shift Keying), PSK (Phase Shift Keying), QPSK (Quadrature Phase Shift Keying), and 16QAM (16 Quadrature Amplitude Modulation).

The wireless communication unit 14 transmits the sample data to the diagnostic apparatus body 10 through wireless communication with the diagnostic apparatus body 10, receives various control signals from the diagnostic apparatus body 10, and outputs the received control signals to the communication controller 18. The communication controller 18 controls the wireless communication unit 14 so that the sample data is transmitted with a transmission wave intensity that is set by the probe controller 19 and outputs various control signals received by the wireless communication unit 14 to the probe controller 19.

The probe controller 19 controls various components of the ultrasound probe 1 according to control signals transmitted from the diagnostic apparatus body 10.

The battery 5 functions as power supply unit of the ultrasound probe 1 to supply the components mounted on the signal processing board 4 and the wireless communication board 5. The battery controller 20 controls the power supply from the battery 6 to the inside of the ultrasound probe 1.

The ultrasound probe 1 uses a scan method of linear scan type, convex scan type, sector scan type, and the like.

On the other hand, the diagnostic apparatus body 10 comprises a wireless communication unit 21, which is connected to a data storage unit 23 via a serial/parallel converter 22. The data storage unit 23 is connected to an image producer 24. The image producer 24 is connected to a monitor 26 via a display controller 25. The wireless communication unit 21 is also connected to a communication controller 27; the serial/parallel converter 13, the image producer 24, the display controller 25, and the communication controller 27 are connected to an apparatus controller 28. The apparatus controller 28 is connected to an operating unit 29 for an operator to perform input operations and a storage unit 30 for storing operation programs.

The wireless communication unit 21 transmits various control signals to the ultrasound probe 1 through wireless communication with the ultrasound probe 1. The wireless communication unit 21 demodulates the signal received by the antenna to output serial sample data.

The communication controller 27 controls the wireless communication unit 21 so that various control signals are transmitted with a transmission radio wave intensity that is set by the apparatus controller 28.

The serial/parallel converter 22 converts the serial sample data outputted from the wireless communication unit 21 into parallel sample data. The data storage unit 23 is configured by a memory, a hard disk, or the like and stores at least one frame of sample data converted by the serial/parallel converter 22.

The image producer 24 performs reception focusing process on every frame of sample data read out from the data storage unit 23 to generate an image signal representing an ultrasound diagnostic image. The image producer 24 comprises a phasing adder 31 and an image processor 32.

The phasing adder 31 selects one reception delay pattern from the plurality of previously stored reception delay patterns according to the reception direction set in the apparatus controller 28 and, based on that selected reception delay pattern, provides the complex base band signals represented by the sample data with respective delays before adding them to perform the reception focusing process. By this reception focusing, a base band signal (sound ray signal) where the ultrasonic echoes are well focused is generated.

The image processor 32 generates a B-mode image signal, which is tomographic image information on a tissue inside the subject, according to the sound ray signal generated by the phasing adder 31. The image processor 32 comprises an STC (sensitivity time control) and a DSC (digital scan converter). For the sound ray signal, the STC corrects attenuation due to distance according to the depth of the reflection position of the ultrasonic waves. The DSC converts the sound ray signal corrected by the STC into an image signal (raster conversion) compatible with the scanning method of an ordinary television signal and performs required image processing, such as contrast processing, to generate a B mode image signal.

The display controller 25 causes the monitor 26 to display an ultrasound diagnostic image according to the image signal generated by the image producer 24. The display unit 26 comprises a display device such as an LCD, for example, and displays an ultrasound diagnostic image under the control of the display controller 25.

The apparatus controller 28 controls the components in the diagnostic apparatus 10 according to various instruction signals and the like entered by the operator using the operating unit 29.

In such diagnostic apparatus body 10, while the serial/parallel converter 22, the image producer 24, the display controller 25, the communication controller 27, and the apparatus controller 28 are each constituted by a CPU and an operation program for causing the CPU to perform various kinds of processing, they may be constituted by a digital circuit. The aforementioned operation program is stored in the storage unit 30. The recording medium in the storage unit 30 may be a flexible disk, MO, MT, RAM, CD-ROM, DVD-ROM or the like besides a built-in hard disk.

Next, the operation of Embodiment 1 will be described.

First, as illustrated in FIGS. 2 and 3, the operator holds the housing 2 from the given direction D in which the projection 8 projects to start a diagnosis with the ultrasound transmission/reception surface 3a of the transducer array 3 housed in the transducer array compartment 7 of the housing 2 in contact with the surface of the subject. As described above, the center of gravity G of the whole ultrasound probe 1 lies on the side of the center line C of the arrangement of the transducer array 3 which is closer to the grip, that is, the center of gravity G lies in a position away from the center line C in the given direction D when the ultrasound transmission/reception surface 3a of the transducer array 3 is placed in a horizontal posture. Thus, the operator holds the grip 9 in such a manner as to embrace the center of gravity G, with the projection 8 housing the battery 6 as a weight positioned above the hand. Therefore, the burden on the operator is lessened, and the operator can hold the ultrasound probe 1 with a significantly increased stability, so that the operator can keep the ultrasound probe 1 in a stable posture with respect to the subject even for a long period of time.

In diagnosis, ultrasonic waves are transmitted from the transducers 11 constituting the transducer array 3 according to the actuation signals that are supplied from the transmission actuator 15 of the ultrasound probe 1, and the reception signals outputted from the transducers 11 that have received the ultrasonic echoes from the subject are supplied to the corresponding reception signal processors 12 to generate sample data, which undergoes conversion into serial data by the parallel/serial converter 13 and then are transmitted wirelessly from the wireless communication unit 14 to the diagnostic apparatus body 10. The sample data received by the wireless communication unit 21 of the diagnostic apparatus body 10 is converted into parallel data through the serial/parallel converter 22 and stored in the data storage unit 23. Further, the data storage unit 23 reads out the sample data by frame, and the image producer 24 generates the image signal and, based on this image signal, the display controller 25 controls the monitor 26 to display the ultrasound diagnostic image.

Thus, according to Embodiment 1, the stability with which the ultrasound probe 1 is held is improved, and the operator burden is reduced so that even an operator who is not an experienced technician can hold the ultrasound probe 1 in a stable posture when giving ultrasound diagnosis and obtain a high accuracy ultrasound image.

### Embodiment 2

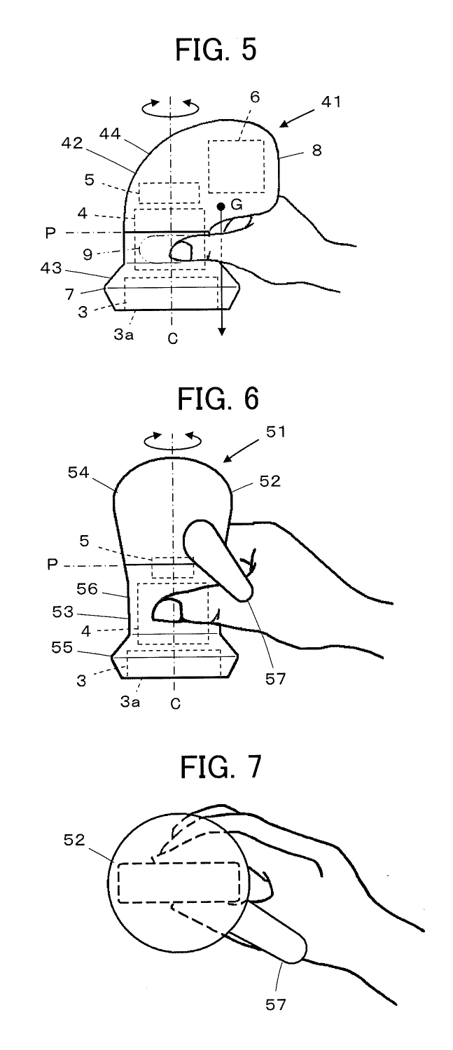

FIG. 5 illustrates an ultrasound probe 41 according to Embodiment 2 of the invention. The ultrasound probe 41 uses a housing 42 instead of the housing 2 in the ultrasound probe 1 of Embodiment 1. The whole shape of the housing 42 is similar to that of the housing 2 except that the former is divided into a first housing 43 comprising the transducer array compartment 7 and the grip 9 and a second housing 44 comprising the projection 8. The second housing 44 is provided so as to be rotatable with respect to the first housing 43 in a plane P substantially parallel to the ultrasound transmission/reception surface 3a of the transducer array 3 about the center line C of the transducer array 3.

As illustrated in FIG. 5, a part of the signal processing board 4 is located in the second housing 44, but the signal processing board 4 does not rotate with the second housing 44 even when the second housing 44 is rotated with respect to the first housing 43, because the signal processing board 4 is secured to the grip 9 of the first housing 43. This is because the signal processing board 4 comprises a circuit for processing the reception signal received by the transducer array 3 and it is therefore preferable to maintain a high-accuracy electric connection between the transducer array 3 and the signal processing board 4.

The wireless communication board 5 may be secured to the grip 9 of the first housing 43 together with the signal processing board 4 so as not to be interfered by the rotation of the second housing 44, or may be secured to the second housing 44 so as to rotate with the second housing 44.

The battery 6 housed in the projection 8 rotates with respect to the first housing 43 as the second housing 44 rotates.

Thus, with the housing 42 divided into the first housing 43 and the second housing 44 so as to be rotatable with respect to each other, the stability of the hold of the ultrasound probe 41 varies with the rotation angle of the second housing 44 containing the battery 6 as a weight with respect to the first housing 43 formed with the grip 9. Therefore, in cases where the manner in which the operator holds the ultrasound probe 1 varies with the operator, who may, for example, holds the probe at an angle, in cases where diagnosis is given with the ultrasound transmission/reception surface 3a of the transducer array 3 slanted or positioned in an upright posture instead of in a horizontal posture, and in other like cases, diagnosis can be given with the second housing 44 rotated to a rotation angle that provides a highest stability as individual operators hold the ultrasound probe 1.

Therefore, diagnosis can be given with the ultrasound probe 1 kept to a stable posture regardless of the manner in which the operator holds the ultrasound probe 1, the posture in which the ultrasound probe 1 is used, and the like.

The housing 42 preferably has a mechanism that, when the second housing 44 is rotated with respect to the first housing 43, maintains a selected rotation angle with a given force by, for example, allowing the rotation angle to be changed incrementally by a given angle. The second housing 44 may be locked to the first housing 43 at a desired rotation angle with a snapping mechanism or a set screw.

Although according to the embodiment 2, the second housing 44 is provided so as to be rotatable with respect to the first housing 43 about the center line C of the transducer array 3, the invention is not limited thereto, provided that the rotation is achieved in the plane P substantially parallel to the ultrasound transmission/reception surface 3a of the transducer array 3.

### Embodiment 3

FIGS. 6 and 7 illustrate an ultrasound probe 51 according to Embodiment 3 of the invention. Similarly to the housing 42 of Embodiment 2, a housing 52 used for the ultrasound probe 51 comprises a first housing 53 and a second housing 54 provided so as to be rotatable with respect to the first housing 53 in the plane P substantially parallel to the ultrasound transmission/reception surface 3a of the transducer array 3.

The first housing 53 comprises a transducer array compartment 55 for housing the transducer array 3 and a grip 56 located above the transducer array compartment 55 and connected and secured to the transducer array compartment 55.

In the grip 56, the signal processing board 4 is disposed immediately above and close to the transducer array 3 and the wireless communication board 5 is disposed above the signal processing board 4. The signal processing board 4 and the wireless communication board 5 are located generally on the center line C of the arrangement of the transducer array 3 and secured to the grip 56 of the first housing 53, so that even when the second housing 54 is rotated with respect to the first housing 53, the signal processing board 4 and the wireless communication board 5 do not rotate with the second housing 54.

The second housing 54 is formed with a projection 57. Unlike the projection 8 in Embodiments 1 and 2, the projection 57 extends not only in a direction perpendicular to the center line C of the arrangement of the transducer array 3 but downward, i.e., to the side closer to the first housing 53 so as to project obliquely downward. Such projection 57 contains a battery serving as a weight, not shown, so that when the ultrasound transmission/reception surface 3a of the transducer array 3 is placed in a horizontal posture, the center of gravity G of the whole ultrasound probe 51 lies in a position outwardly away from the center line C of the arrangement of the transducer array 3

As the operator holds the grip 56 of the first housing 53 as illustrated in FIG. 6, the projection 57 housing the battery as a weight is located above the hand of the operator because the projection 57 projects obliquely downward, and thus the operator can hold the ultrasound probe 1 with an increased stability. Further, even when the operator's holding force weakens for some reason during diagnosis or the operator's hand slips from the housing 52, the projection 57 catches a finger of the operator and thereby reduces the possibility of the ultrasound probe 1 falling.

Further, since the housing 52 is divided into the first housing 53 and the second housing 54 so as to be rotatable with respect to each other, diagnosis can be given with the ultrasound probe 51 kept to a stable posture similarly to Embodiment 2 regardless of the manner in which the operator holds the ultrasound probe 1, the posture in which the ultrasound probe 1 is placed, and the like.

In a case where the projection 57 does not have enough space to house the battery, the battery may be disposed in a region inside the second housing 54 other than the projection 57 while a weight may be provided inside the projection 57 in order to position the center of gravity G of the whole ultrasound probe 51 outwardly away from the center line C of the arrangement of the transducer array 3.

Although the ultrasound probes 1, 41, and 51 and the diagnostic apparatus body 10 are connected to each other by wireless communication in Embodiments 1 to 3, the invention is not limited thereto and the ultrasound probes 1, 41, and 51 may be connected to the diagnostic apparatus body 10 via a connection cable. In such a case, the wireless communication board 5 having the wireless communication unit 14 and the communication controller 18 mounted thereon and the battery 6 are unnecessary in the ultrasound probes 1, 41, and 51 as well as the wireless communication unit 21 and the communication controller 27 in the diagnostic apparatus 10. Thus, some weight instead of the battery 6 may be provided inside the projections 8 and 57.

What is claimed is:

1. An ultrasound probe comprising:
a transducer array having an ultrasound transmission/reception surface;
a processing circuit mounted on a signal processing board for processing an ultrasonic echo from a subject received with the transducer array to produce a reception signal;
a housing including a transducer array compartment for housing the transducer array at one end and the signal processing board near the transducer array,
the housing including a projection at another end projecting in a given direction perpendicular to a center line of an arrangement of the transducer array and a grip between the projection and the transducer array compartment for holding from the given direction,
a center of gravity lying in a position away from the center line of the arrangement of the transducer array in the given direction when the ultrasound transmission/reception surface of the transducer array is in a horizontal posture.

2. The ultrasound probe according to claim 1, wherein the center of gravity lies on an outside of a periphery of the grip in the given direction when the ultrasound transmission/reception surface of the transducer array is in a horizontal posture.

3. The ultrasound probe according to claim 1, wherein a weight is housed inside the projection.

4. The ultrasound probe according to claim 3, further comprising:
a wireless communication board having a wireless communication circuit mounted thereon for wireless communication with a diagnostic apparatus body, the wireless communication board being contained inside the housing; and
a battery for supplying power to the processing circuit mounted on the signal processing board and the wireless communication circuit mounted on the wireless communication board.

5. The ultrasound probe according to claim 4, wherein the battery is contained inside the projection as the weight.

6. The ultrasound probe according to claim 1, wherein the projection is provided so as to be rotatable with respect to the grip in a plane parallel to the ultrasound transmission/reception surface of the transducer array.

7. The ultrasound probe according to claim 6, wherein the signal processing board is secured to the grip.
